(19) United States
(12) Patent Application Publication
(10) Pub. No.: US 2018/0335421 A1
(43) Pub. Date: Nov. 22, 2018

(54) METHOD FOR SCREENING INHIBITORS TARGETING ANTI-APOPTOTIC SURVIVAL PATHWAYS

(71) Applicant: Memorial Sloan-Kettering Cancer Center, New York, NY (US)

(72) Inventors: Emily H. Cheng, Englewood Cliffs, NJ (US); James Hsieh, Englewood Cliffs, NJ (US)

(73) Assignee: Memorial Sloan-Kettering Cancer Center, New York, NY (US)

(21) Appl. No.: 15/776,596

(22) PCT Filed: Nov. 18, 2016
(86) PCT No.: PCT/US16/62789

§ 371 Date: May 16, 2018

Related U.S. Application Data
(60) Provisional application No. 62/258,386, filed on Nov. 20, 2015.

(51) Int. Cl. G01N 33/50; A61K 31/343; A61P 35/00

(52) U.S. Cl. CPC G01N 33/5011; G01N 2510/00; A61P 35/00; A61K 31/343

(57) ABSTRACT

A method of identifying inhibitors of the anti-apoptotic survival pathway in cancer cells is disclosed. The method comprises the steps of (a) exposing cultured wild-type cells to a candidate inhibitor at a predetermined concentratioh for a predetermined period of time and determining cell viability aftet the exposure to the candidate inhibitor; (b) exposing two or more cell lines of specifically MCL-1 or BCL-2 or BCL-X₁ addicted cells to the candidate inhibitor at the predetermined concentration for the predetermined period of time and determining cell viability after the exposure to the candidate inhibitor, and (c) indentifying the candidate inhibitor as a MCL-1 or BCL-2 or BCL-X₁ inhibitor if the cell viability in step (a) is significantly higher than the cell viability in step (b). The disclosed method provides a way to identify inhibitors which selectively inhibit specific members of the BCL-2 family (e.g., MCL-1) by screening two or more cell lines with addictions to different and specific members of the BCL-2 family of proteins.

### RELATED APPLICATIONS

The present application claims priority under 35 U.S.C. § 119(e) to U.S. Provisional Patent Application, U.S.S.N. 62/258,383, filed Nov. 20, 2015, which is incorporated herein by reference.

### BACKGROUND OF THE INVENTION

BCL-2, BCL-X_{L} and MCL-1 are anti-apoptotic members of the BCL-2 family that govern cellular commitment to apoptosis at the mitochondria. Overexpression of the anti-apoptotic BCL-2 family proteins contributes to tumor initiation, progression, and resistance to anticancer treatments. Hence, anti-apoptotic BCL-2 proteins are attractive targets for anticancer therapy. In fact, clinical efficacy of the BCL-2/BCL-X_{L} inhibitor, ABT-263 (navitoclax), and the BCL-2-specific inhibitor, ABT-199 (GDC-0199 or venetoclax), have been demonstrated in clinical trials. However, both ABT-263 and ABT-199 fail to inhibit MCL-1, and overexpression of MCL-1 confers resistance to BCL-2/BCL-X_{L} inhibitors. Nonetheless, MCL-1 is the most frequently amplified anti-apoptotic BCL-2 member in human cancers (among the top 10 most frequently amplified genes in all cancer types), and cancer cells with MCL-1 amplification or overexpression are shown to be addicted to MCL-1 for survival. Hence, effective inactivation of the MCL-1-dependent survival pathway could hold great promise for cancer therapy.

Programmed cell death, referred to as apoptosis, plays an indispensable role in the development and maintenance of tissue homeostasis within all multicellular organisms. Dysregulation of apoptosis causes human illness ranging from cancer to neurodegenerative disorders. The BCL-2 family proteins are central regulators of apoptosis. Promotion or induction of apoptosis through inhibition of BCL-2, BCL-X_{L}, or MCL-1 is not only useful for the treatment of cancers but also for the treatment of disorders associated with defective apoptosis such as arthritis, inflammation, lymphoproliferative conditions, and autoimmune diseases. The BCL-2 family is implicated in the pathogenesis of a variety of autoimmune disorders including autoimmune glomerulonephritis, immunoglobulinemia, systemic lupus erythematosus (SLE), type I diabetes mellitus, and multiple sclerosis. Furthermore, some DNA viruses, such as Epstein-Barr virus, African swine fever virus, and adenovirus, parasitize the host cellular machinery to drive their replication and at the same time modulate apoptosis to repress cell death and allow the target cell to reproduce the virus. Hence, induction of apoptosis may limit the infection of these DNA viruses. In addition, because the biochemical features of BCL2A1 are most similar to those of MCL-1, MCL-1 inhibitors likely inhibit BCL2A1 and thereby can be used to treat disease processes caused by aberrant upregulation of BCL2A1. By analogy, BCL-X_{L} inhibitors also likely inhibit BCL-W.

Due to the inherent limitations of using standard cell-free systems for targeting MCL-1, no clinically applicable MCL-1 inhibitors have been developed. The lack of specific MCL-1 inhibitors for clinical use constitutes an unmet need for cancer therapy. Given that MCL-1 is frequently amplified in solid tumors (e.g., TCGA datasets: 15.7% in lung adenocarcinoma including K-RAS mutant lung cancer; 15.5% in hepatocellular carcinoma; 13.4% bladder cancer; and 9.3% in invasive breast carcinoma) and highly expressed in acute myeloid leukemia and multiple myeloma, MCL-1 inhibitors likely offer a new paradigm in targeted cancer therapy.

### SUMMARY OF THE INVENTION

There is a great need for new therapies based on MCL-1 inhibitors; however, the development of small molecule inhibitors of anti-apoptotic BCL-2 family proteins has been mainly focused on in vitro structure-based approaches. Despite the triumph in structure-based discovery of BCL-2 and BCL-X_{L} inhibitors, similar approaches employed in the development of MCL-1 inhibitors have been less successful. To address this weakness, provided herein is a cell death mechanism-guided, cell-based screening strategy for the discovery of MCL-1 inhibitors. While useful for the discovery of MCL-1 inhibitors, the present invention is not limited to the discovery of MCL-1 inhibitors and can also be employed in the discovery of inhibitors of BCL-2, BCL-X_{L}, and other members of the BCL-2 family of proteins.

To evade apoptotic checkpoints, cancer cells often overexpress anti-apoptotic BCL-2 family proteins including BCL-2, BCL-X_{L}, and MCL-1. Counterintuitively, cancer cells also commonly express higher levels of pro-apoptotic BIM (BH3 interacting-domain death agonist) and PUMA (p53 up-regulated modulator of apoptosis). One plausible explanation is that BIM and PUMA are transcriptionally activated by E2F1, a key cell cycle driver upon malignant transformation, and are sequestered by anti-apoptotic BCL-2, BCL-X_{L} or MCL-1 as insert complexes. Hence, many cancer cells are likely “primed” to undergo apoptosis upon the administration of BAD and NOXA mimetics that displace BIM/PUMA from BCL-2/BCL-X_{L} and MCL-1, respectively, to activate the apoptotic gateway BAX and BAK. Using cells that express different combinations of anti-apoptotic BCL-2 members and activator BH3s, such as BIM and PUMA, using a bicistronic internal ribosomal entry site (IRES) vector, the invention provides a system that recapitulates cancers with specific addictions to BCL-2, BCL-X_{L}, or MCL-1, and allows for screening (e.g., high-throughput screening (HTS)) to identify inhibitors of BCL-2, BCL-X_{L}, or MCL-1. This inventive system mimics the “primed” cell death state of many cancers with abundant pre-assembled complexes of anti-apoptotic BCL-2 members and activator BH3s. In addition, cell lines with selective addictions to BCL-2, BCL-X_{L}, or MCL-1 for survival can be engineered, which can be utilized for screening (e.g., HTS) for the discovery of specific inhibitors of anti-apoptotic BCL-2 family proteins.

A limitation of cell-based assays for the identification of BCL-2 family inhibitors is the inability to clearly identify inhibitors which selectively target specific members of the BCL-2 family. As described herein, the present invention can be used to identify mechanism-specific compounds with cellular activity. In some embodiments of the present invention, parallel screens are performed on wild-type and BCL-2 member-addicted cells to identify chemicals that selectively induce apoptosis in the BCL-2 member-addicted cells but not wild-type cells, and a parallel screen is performed using two or more cell lines specifically addicted to different members of the BCL-2 family. For example, in a particular embodiment of the invention, parallel screening can be performed on wild-type and MCL-1-IRES-BIM expressing MEFs (mouse embryonic fibroblasts) to identify chemicals (e.g., small molecules) that selectively induce apoptosis in MCL-1-IRES-BIM but not wild-type cells, and parallel screening can be performed using MCL-1- and BCL-X_{L}-addicted cells.

In one aspect, the present invention provides methods of engineering cells that mimic the “primed” cell death state of many cancers with a specific addiction to anti-apoptotic proteins for survival. In certain embodiments, the method comprises engineering cells that are addicted to one or more members of the BCL-2 family of proteins. In certain embodiments, the method comprises engineering cells that are addicted to BCL-2, BCL-X_{L}, MCL-1, or any combination thereof. In certain embodiments, the method comprises engineering cells that are addicted to MCL-1. In certain embodiments, the method comprises the steps of (a) expressing different combinations of anti-apoptotic BCL-2 members and activator BH3s (e.g., BIM, PUMA) in cells (e.g., mouse embryonic fibroblasts) using a bicistronic internal ribosomal entry site (IRES) system; and (b) converting the addiction of a cancer cell line to a specific anti-apoptotic BCL-2 member for survival to another anti-apoptotic BCL-2 member. For example, certain cells (e.g., H23, a K-RAS mutant lung cancer cell line) are dependent on MCL-1 for survival because knockdown of MCL-1 induces robust apoptosis, and its addiction to MCL-1 can be converted to BCL-2 or BCL-X_{L} addiction by overexpressing BCL-2 or BCL-X_{L} followed by knockdown of MCL-1. Alternatively, addiction to BCL-2 and/or BCL-X_{L} can be converted to MCL-1 addiction by overexpression of MCL-1 followed by knockdown of BCL-2 and/or BCL-X_{L}

In another aspect, the present invention provides methods for identifying inhibitors of anti-apoptotic survival pathways. In some embodiments, the method comprises the steps of (a) exposing cultured wild-type cells to a candidate inhibitor at a predetermined concentration for a predetermined period of time and determining cell viability after the exposure to the candidate inhibitor; (b) exposing BCL-2 member protein (e.g., MCL-1, BCL-2, BCL-X_{L})-addicted cells to the candidate inhibitor at the predetermined concentration for the predetermined period of time and determining cell viability after the exposure to the candidate inhibitor; and (c) identifying the candidate inhibitor as a BCL-2 family inhibitor (e.g., an inhibitor of MCL-1, BCL-2, or BCL-X_{L}) if the cell viability in step (a) is significantly higher than the cell viability in step (b). In certain embodiments, cells expressing MCL-1-IRES-BIM or MCL-1-IRES-PUMA are addicted to MCL-1 for survival and can be utilized in screening for MCL-1 inhibitors. In certain embodiments, cells expressing BCL-2-IRES-BIM or BCL-2-IRES-PUMA are addicted to BCL-2 for survival and can be utilized in screening for BCL-2 inhibitors. In other embodiments, cells expressing BCL-X_{L}-IRES-BIM or BCL-X_{L}-IRES-PUMA are addicted to BCL-X_{L} for survival and can be utilized in screening for BCL-X_{L} inhibitors.

In certain embodiments, the method of screening BCL-2 member inhibitors allows for the identification of compounds which inhibit specific members of the BCL-2 family. For example, in some embodiments, the method comprises the steps of (a) exposing cultured wild-type cells to a candidate inhibitor at a predetermined concentration for a predetermined period of time and determining cell viability after the exposure to the candidate inhibitor; (b) exposing two or more cell lines independently addicted to specific members of the BCL-2 family (e.g., MCL-1 or BCL-2 or BCL-X_{L}) to the candidate inhibitor at the predetermined concentration for the predetermined period of time and determining cell viability after the exposure to the candidate inhibitor; and (c) identifying the candidate inhibitor as a BCL-2 family member (e.g., MCL-1 or BCL-2 or BCL-X_{L}) inhibitor if the cell viability in step (a) is significantly higher than the cell viability in step (b). In certain embodiments, the method further comprises step (d) identifying the candidate inhibitor as a selective inhibitor of a specific BCL-2 member inhibitor (e.g., MCL-1 or BCL-2 or BCL-X_{L}) if the cell viability of one of the cell lines in step (b) is significantly lower than the cell viability of the other cell line(s) in step (b).

No clinically acceptable inhibitors of MCL-1 have been developed thus far, and the present invention allows for screening and identification of such inhibitors. Therefore, in certain embodiments of the present invention, the method of screening inhibitors comprises the steps of (a) exposing cultured wild-type cells to a candidate inhibitor at a predetermined concentration for a predetermined period of time and determining cell viability after the exposure to the candidate inhibitor; (b) exposing MCL-1 addicted cells and BCL-2 or BCL-X_{L} addicted cells to the candidate inhibitor at the predetermined concentration for the predetermined period of time and determining cell viability after the exposure to the candidate inhibitor; and (c) identifying the candidate inhibitor as a MCL-1 inhibitor if the cell viability in step (a) is significantly higher than the cell viability in step (b). In certain embodiments, the method further comprises step (d) identifying the candidate inhibitor as a selective inhibitor of MCL-1 if the cell viability of the BCL-2 or BCL-X_{L}-addicted cells is significantly higher than the cell viability of the cell viability of the MCL-1 addicted cells in step (b).

Extent of apoptosis can also be a measure of a candidate inhibitor's activity as a BCL-2 family inhibitor. In some embodiments, the extent of apoptosis can be assessed by caspase activity. Therefore, in certain embodiments, the method of screening inhibitors comprises the steps of (a) exposing cultured wild-type cells to a candidate inhibitor at a predetermined concentration for a predetermined period of time and determining extent of apoptosis after the exposure to the candidate inhibitor; (b) exposing MCL-1 addicted cells and BCL-2 or BCL-X_{L} addicted cells to the candidate inhibitor at the predetermined concentration for the predetermined period of time and determining extent of apoptosis after the exposure to the candidate inhibitor; and (c) identifying the candidate inhibitor as a MCL-1 inhibitor if the extent of apoptosis in step (b) is significantly higher than the cell viability in step (a). In certain embodiments, the method further comprises step (d) identifying the candidate inhibitor as a selective inhibitor of MCL-1 if the extent of apoptosis of the MCL-1 addicted cells is significantly higher than the extent of apoptosis of the BCL-2 or BCL-X_{L}-addicted cells in step (b). Apoptosis is as defined herein.

The present invention also provides cell lines for carrying out the methods described herein, as well as kits comprising the cell lines. The cell lines can comprise cells that are addicted to one or more members of the BCL-2 family of proteins (e.g., MCL-1, BCL-2, BCL-X_{L}).

Described herein are methods of screening for BCL-2 family inhibitors which may be useful in treating cancer. Also provided herein are methods of treating diseases with BCL-2 family inhibitors. In certain embodiments, the method comprises administering to a subject in need thereof an effective amount of a BCL-2 family inhibitor, or a pharmaceutically selective salt thereof, or a pharmaceutical composition thereof. In some embodiments, the method comprises administering to a subject in need thereof an effective amount of a selective MCL-1 inhibitor, or a pharmaceutically acceptable salt thereof, or a pharmaceutical composition thereof.

### Definitions

As used herein, “BCL-2 family” refers to the apoptosis regulator BCL-2 family, a family of evolutionary-related proteins that regulate apoptosis in cells mainly by regulating the outer mitochondrial membrane integrity (see, e.g., Czabotar et al., Nat. Rev. Mol. Cell Biol., 2014, 15, 49-63). BCL-2 family proteins can be “pro-apoptotic” (e.g., BAX, BAD, BAK, BOK) or “anti-apoptotic” (e.g., parent BCL-2, BCL-X_{L}, BCL-W, MCL-1). Proteins of anti-apoptotic BCL-2 subfamily have up to four BH (BCL-2 homology) domains named BH1-4, and prevent cells from entering apoptosis. The BCL-2 pro-apoptotics can be further grouped into the multidomain pro-apoptotic and BH3-only proteins. The multidomain pro-apoptotic effectors, BAX and BAK, also contain four BH (BH1-4) regions and promote cell death by oligomerization-mediated mitochondria outer membrane permeabilization (MOMP). The BH3-only proteins share the BH3 region of sequence similarity. Members of this group include BID, BIM, BAD, BMF, BIK, PUMA, NOXA, HRK/DP5 (Harakiri), NIX, and BNIP3. The BH3 domain is 16 to 25 amino acid residues long and some BH3 peptides can promote apoptosis when introduced into cells. The three groups of BCL-2 family proteins form a delicately balanced network of opposing functions that regulates the cell's fate. In certain embodiments, the BCL-2 is a BCL-2 anti-apoptotic. In certain embodiments, the BCL-2 anti-apoptotic is BCL-2, BCL-W, BCL-X_{L}, or MCL-1. In certain embodiments, the BCL-2 anti-apoptotic is MCL-1. In certain embodiments, compounds may interact (e.g., inhibit or activate) with at least one anti-apoptotic protein member of the BCL-2 family, thereby enhancing apoptosis. In certain embodiments, compounds may interact with at least one anti-apoptotic protein member of the BCL-2 family and induce its degradation. In certain embodiments, compounds may interact with at least one pro-apoptotic protein member of the BCL-2 family, thereby enhancing apoptosis. Proteins that belong to the BCL-2 family may be referred to as “BCL-2 members” or “BCL-2 family members”. Proteins that belong to the multidomain BCL-2 family include, but are not limited to BAK (BAK1), BAX, parent BCL-2, A1 (BCL2A1), BCL-XL (BCL2L1), BCL-W (BCL2L2), BCL-B (BCL2L10), BCL-RAMBO (BCL2L13), BCL-G (BCL2L14), BOK, and MCL-1. As used herein, “BCL-2” or “parent BCL-2” refers to B-cell lymphoma 2, an anti-apoptotic member of the BCL-2 family which helps regulate apoptosis in cells. As used herein, “BCL-X_{L}” refers to B-cell lymphoma-extra long. As used herein, “MCL-1” refers to induced myeloid leukemia cell differentiation protein MCL-1. Any isoforms of the BCL-2 family proteins described herein are contemplated as being within the scope of the invention.

As used herein, the term “addicted” refers to a cell's dependence on an anti-apoptotic protein for survival. For example, a cell is addicted to an anti-apoptotic protein if the anti-apoptotic protein regulates apoptosis (i.e., programmed cell death) in the cell. In some instances, cells can be addicted to anti-apoptotic BCL-2 family member proteins for survival, and the BCL-2 family member proteins mitigate apoptosis in the cell. In some instances, a cell's addiction to a protein coincides with overexpression of the protein or predominant expression of the protein versus other related proteins in the cell. In some instances, a cell that is addicted to one or more BCL-2 family member proteins (e.g., BCL-2, BCL-X_{L}, MCL-1) has one or more of the proteins overexpressed and/or predominantly expressed in the cell.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

As used herein, the term “apoptosis” refers to a regulated network of biochemical events which lead to a selective form of cell suicide and is characterized by readily observable morphological and biochemical phenomena. Cells undergoing apoptosis show characteristic morphological and biochemical features. These features include chromatin aggregation or condensation, DNA fragmentation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial outer membrane permeabilization accompanying apoptosis.

As used herein, “isogenic” refers to cells that are selected or engineered to model a disease. For example, isogenic cancer cells are cells that have been selected or engineered to model cancer cells.

As used herein, “inhibition”, “inhibiting”, “inhibit” and “inhibitor”, and the like, refer to the ability of a compound to reduce, slow, halt, or prevent the activity of an anti-apoptotic BCL-2 family protein (also called “pro-survival BCL-2 family protein”). In certain embodiments, such inhibition is of about 1% to 99.9%. In certain embodiments, the inhibition is about 1% to about 95%. In certain embodiments, the inhibition is about 5% to 90%. In certain embodiments, the inhibition is about 10% to 85%. In certain embodiments, the inhibition is about 15% to 80%. In certain embodiments, the inhibition is about 20% to 75%. In certain embodiments, the inhibition is about 25% to 70%. In certain embodiments, the inhibition is about 30% to 65%. In certain embodiments, the inhibition is about 35% to 60%. In certain embodiments, the inhibition is about 40% to 55%. In certain embodiments, the inhibition is about 45% to 50%. In certain embodiments, the inhibition is about 5%, 10%, 15%, 20%, 25%, 30%, 35%, 40%, 45%, 50%, 55%, 60%, 65%, 70%, 75%, 80%, 85%, 90%, 95%, or 99.9%.

When a compound is referred to as “selectively” inhibiting (i.e., when a compound is referred to as a “selective inhibitor” of) a specific BCL-2 family protein, the compound inhibits the specific BCL-2 family protein to a greater extent (e.g., more than 1-fold, not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than it inhibits a different BCL-2 family protein. A selective MCL-1 inhibitor (i.e., a compound that selectively inhibits MCL-1) inhibits the MCL-1 to a greater extent (e.g., more than 1-fold, not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than it inhibits a different BCL-2 family protein.

In certain embodiments, the candidate inhibitors described herein can act as NOXA mimetics. “NOXA” is a pro-apoptotic BH3-only member of the BCL-2 protein family that specifically inactivates MCL-1 and has been shown to be involved in p53-mediated apoptosis. In certain embodiments, the candidate inhibitors described herein mimic NOXA and bind to the hydrophotic dimerization groove of MCL-1 and induce apoptosis in MCL-1 addicted cancer cells. In certain embodiments, the candidate inhibitors described herein induce the degradation of MCL-1 and thereby trigger apoptotis in MCL-1 addicted cancer cells.

“High throughput screening” (i.e., “HTS”) refers to a method of screening that relies automation to rapidly assay the biological activity of multiple agents. Typically, HTS screening involves conducting multiple assays in parallel in order to quickly identify agents that successfully modulate a certain biomolecular pathway. In certain embodiments, HTS involves conducting multiple assays (i.e., more than three) in parallel. In certain embodiments, HTS involves running more than 50 assays in parallel. In certain embodiments, HTS involves running more 100 assays in parallel. In certain embodiments, HTS involves running more than 500 assays in parallel. In certain embodiments, HTS involves running more than 1,000 assays in parallel. In certain embodiments, HTS involves running more than 10,000 assays in parallel. In certain embodiments, HTS involves running more than 50,000 assays in parallel. In certain embodiments, HTS involves running more than 100,000 assays in parallel. “Low-throughput screening” (i.e., “LTS”) refers to a method of screening agents that is not high-throughput.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a BCL-2 family inhibitor (e.g., MCL-1 inhibitor) refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g.,bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, diseases associated with angiogenesis or diseases associated with angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “angiogenesis” refers to the formation and growth of new blood vessels. Normal angiogenesis occurs in the body of a healthy subject during wound healing and for restoring blood flow to tissues after injury. The body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal angiogenesis refers to angiogenesis greater than that in a normal body, especially angiogenesis in an adult not related to normal angiogenesis (e.g., menstruation or wound healing). Abnormal angiogenesis can result in new blood vessels that feed diseased tissues and/or destroy normal tissues, and in the case of cancer, the new vessels can allow tumor cells to escape into the circulation and lodge in other organs (tumor metastases). In certain embodiments, the disease associated with angiogenesis is tumor angiogenesis. In certain embodiments, the diseases associated with angiogenesis include, but are not limited to breast cancer, colorectal cancer, esophageal cancer, gastrointestinal stromal tumor (GIST), kidney (renal cell) cancer, liver (adult primary) cancer, lymphoma, melanoma, lung cancer, ovarian epithelial cancer, pancreatic cancer, prostate cancer, stomach (gastric) cancer.

As used herein, an “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyosifis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomylitis, optic neuritis, temporal arteritis, transverse myelitis, necrotizing fascilitis, and necrotizing enterocolitis. In certain embodiments, the inflammatory disease is arthritis.

As used herein, an “autoimmune disease” refers to a disease arising from an inappropriate immune response in the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppressants, e.g., medications which decrease the immune response. Exemplary autoimmune diseases include, but are not limited to, glomerulonephritis, Goodspature's syndrome, necrotizing vasculitis, lymphadenitis, peri-arteritis nodosa, systemic lupus erythematosis, rheumatoid, arthritis, psoriatic arthritis, systemic lupus erythematosis, psoriasis, ulcerative colitis, systemic sclerosis, dermatomyositis/polymyositis, anti-phospholipid antibody syndrome, scleroderma, perphigus vulgaris, ANCA-associated vasculitis (e.g., Wegener's granulomatosis, microscopic polyangiitis), urveitis, Sjogren's syndrome, Crohn's disease, Reiter's syndrome, ankylosing spondylitis, Lyme arthritis, Guillain-Barre syndrome, Hashimoto's thyroiditis, and cardiomyopathy. In certain embodiments, the autoimmune disease is autoimmune glomerulonephritis, immunoglobulinemia, or systemic lupus erythematosus (SLE).

The term “autoinflammatory disease” refers to a category of diseases that are similar but different from autoimmune diseases. Autoinflammatory and autoimmune diseases share common characteristics in that both groups of disorders result from the immune system attacking a subject's own tissues and result in increased inflammation. In autoinflammatory diseases, a subject's innate immune system causes inflammation for unknown reasons. The innate immune system reacts even though it has never encountered autoantibodies or antigens in the subject. Autoinflammatory disorders are characterized by intense episodes of inflammation that result in such symptoms as fever, rash, or joint swelling. These diseases also carry the risk of amyloidosis, a potentially fatal buildup of a blood protein in vital organs. Autoinflammatory diseases include, but are not limited to, familial Mediterranean fever (FMF), neonatal onset multisystem inflammatory disease (NOMID), tumor necrosis factor (TNF) receptor-associated periodic syndrome (TRAPS), deficiency of the interleukin-1 receptor antagonist (DIRA), and Behçet's disease.

“Anti-cancer agents” and “anti-proliferative agents” encompass biotherapeutic agents as well as chemotherapeutic agents. Exemplary biotherapeutic anti-cancer agents include, but are not limited to, interferons, cytokines (e.g., tumor necrosis factor, interferon a, interferon y), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g., hydroxyurea and deferoxamine), uracil analogs (e.g. 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g. mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g. actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. In certain embodiments, the additional anti-cancer agent is an inhibitor of BCL-2. In certain embodiments, the additional anti-cancer agent is an inhibitor of BCL-X_{L}. In certain embodiments, the additional anti-cancer agent is an inhibitor of an anti-apoptotic BCL-2 family protein. In certain embodiments, the additional anti-cancer agent is navitoclax, 4-[4-[[2-(4-Chlorophenyl)-5,5-dimethyl-1-cyclohexen-1-yl]methyl]-1-piperazinyl]-N-[[4-[(1R)-3-(4-morpholinyl)-1-[(phenylthio)methyl]propyl]amino]-3-[(trifluoromethyl)sulfonyl]phenyl]sulfonyl]benzamide (ABT-263), (R)-4-(4-((4′-chloro-[1,1′-biphenyl]-2-yl)methyl)piperazin-1-yl)-N-((4-((-4-(dimethylamino)-1-(phenylthio)butan-2-yl)amino)-3-nitrophenyl)sulfonyl)benzamide (ABT-737), venetoclax (ABT-199), 1,1′,6,6′,7,7′-hexahydroxy-5,5′-diisopropyl-3,3′-dimethyl-[2,2′-binaphthalene]-8,8′-dicarbaldehyde (AT-101), (Z)-2-(2-((3,5-dimethyl-1H-pyrrol-2-yl)methylene)-3-methoxy-2H-pyrrol-5-yl)-1H-indole methanesulfonate (GX15-070), 5-(2-isopropylbenzyl)-N-(4-(2-tert-butylphenylsulfonyl)phenyl)-2,3,4-trihydroxybenzamide (TW-37), Gossypol, (−)-epigallocatechin gallate, obatoclax mesylate, licochalcone A, HA14-1, EM20-25, nilotinib, YC137, 2-methoxy-antimycin A3, ABT-199, gambogic Acid, or nilotinib. In certain embodiments, the additional anti-cancer agent is an inhibitor of MCL-1. In certain embodiments, the additional anti-cancer agent is ABT-263. In certain embodiments, the additional anti-cancer agent is ABT-199.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the system for hierarchical regulation of mitochondrion-dependent apoptosis by BCL-2 subfamilies.

FIGS. 2A-2B. FIG. 2A depicts selective inhibition of BCL-2/BCL-X_{L} and MCL-1 by BAD mimetics and NOXA mimetics, respectively. FIG. 2B shows that BAD displaces BIM/PUMA from BCL-2 or BCL-X_{L} whereas NOXA displaces BIM/PUMA from MCL-1 to activate BAX- and BAK-dependent apoptosis.

FIGS. 3A-3B. FIG. 3A shows H23 is addicted to MCL-1 for survival because knockdown of MCL-1 induces apoptosis in H23 but not A549 cells. Isogenic H23 cancer cell lines with selective addiction to MCL-1, BCL-2, or BCL-X_{L}. FIG. 3B shows that selective addiction of engineered H23 cells was confirmed by treating these cell lines with inhibitors of BCL-2 (ABT-737 and ABT-199), BCL-X_{L} (ABT-737), and MCL-1 (F9). As expected, the BCL-2-addicted H23 cells are sensitive to ABT-737 and ABT-199 but not inhibitors of MCL-1, the BCL-X_{L}-addicted H23 cells are sensitive to ABT-737 but not ABT-199 or inhibitors of MCL-1, and parental H23 cells are only sensitive to inhibitors of MCL-1. The structure of compound F9 is shown.

FIG. 4 shows BCL-2 Family: 3 Subfamilies, including the anti-apoptosis (“Anti-Death”) subfamily of the BCL-2 family, which includes BCL-2, BCL-X_{L}, MCL-1, A1 (BCL2A1), and BCL-W.

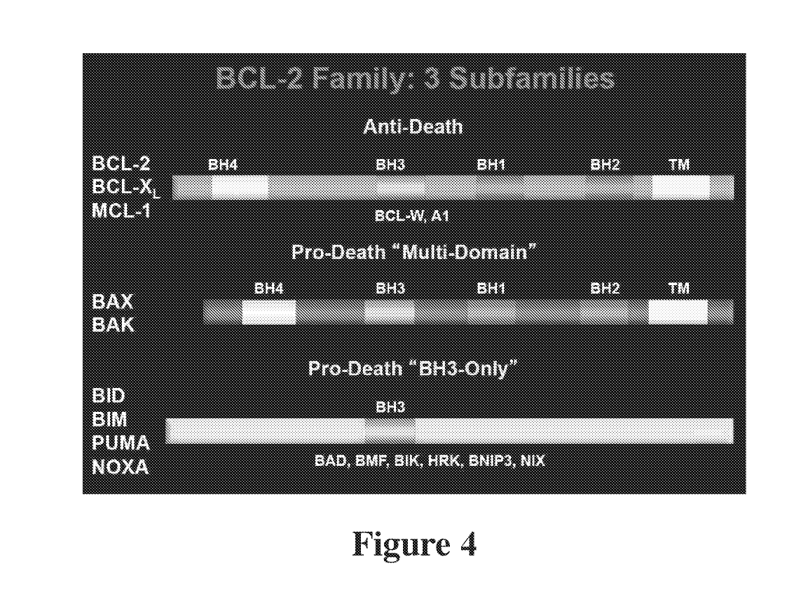

FIG. 5 shows Death Signals.

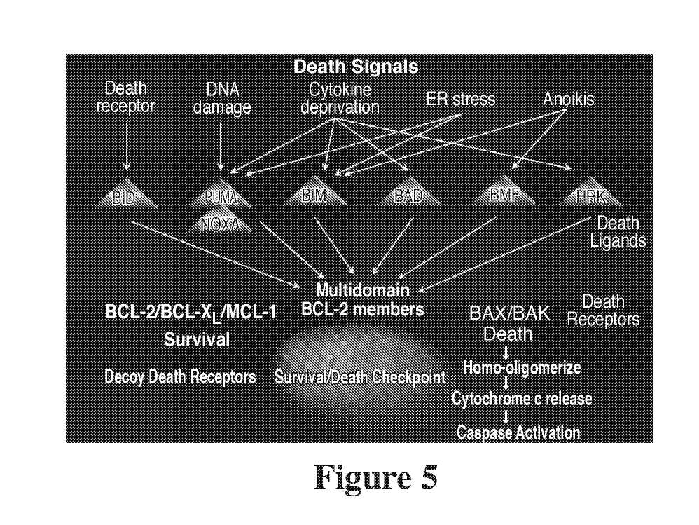

FIG. 6 shows A “Two Class” Model of BH3-Only Molecules.

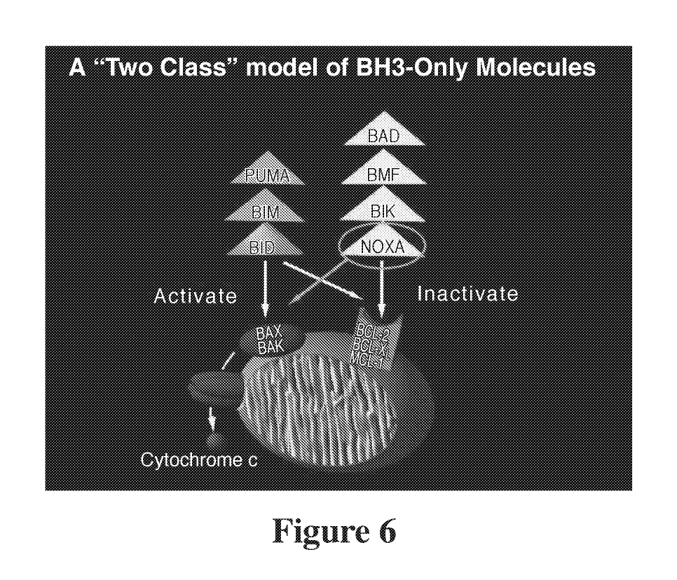

FIG. 7 shows A BAD mimetic or ABT-737/263 displaces BIM/PUMA from BCL-2/BCL-XL to activate BAX/BAK and induce apoptosis.

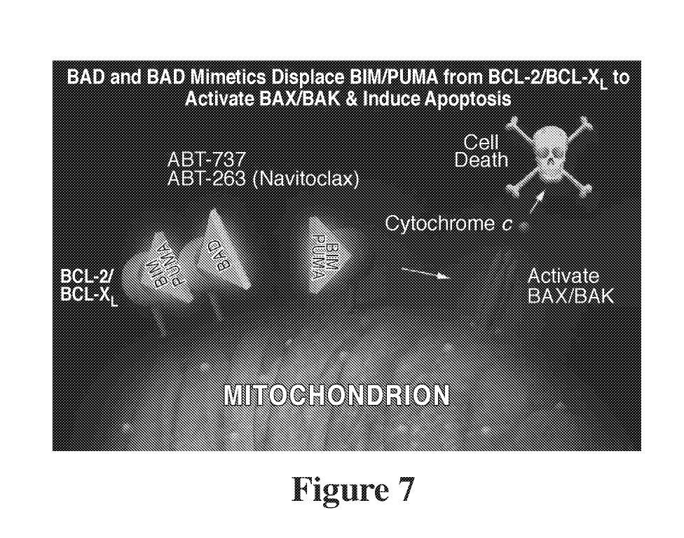

FIG. 8 shows a BAD mimetic or ABT-737/263 is not able to displace BIM/PUMA from MCL-1 to Activate BAX/BAK.

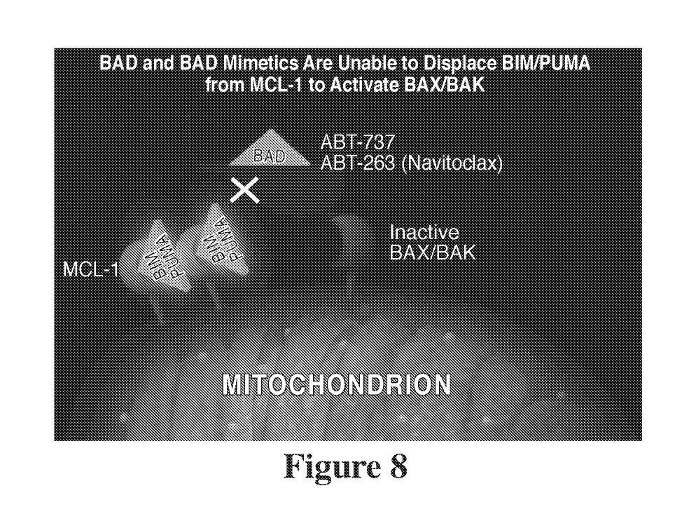

FIG. 9 shows a NOXA mimetic displaces BIM/PUMA from MCL-1 to activate BAX/BAK and induce apoptosis.

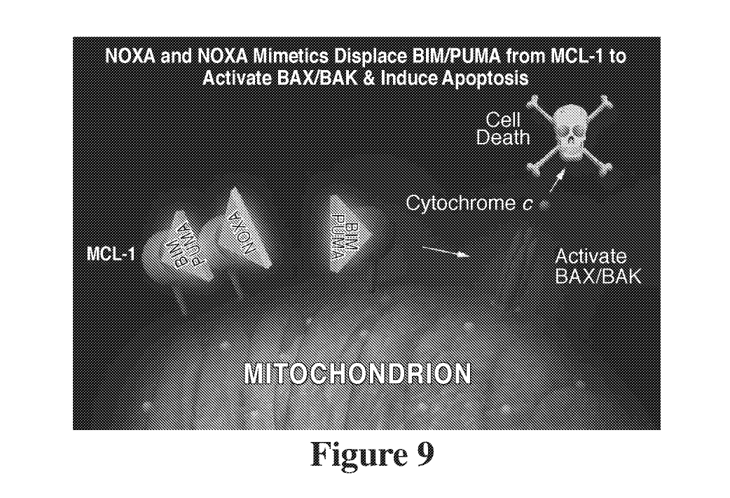

FIG. 10 shows a cell-based screening strategy to identify MCL-1 inhibitors. MEFs expressing MCL-1-IRES-BIM are addicted to MCL-1 for survival whereas wild-type MEFs are not addicted to any single anti-apoptotic BCL-2 member for survival. According, MCL-1 inhibitors, such as NOXA mimetics, can displace BIM from MCL-1 to activate BAX- and BAK-dependent apoptosis in MEFs expressing MCL-1-IRES-BIM but not in wild-type MEFs. Accordingly, wild-type MEFs and MEFs expressing MCL-1-IRES-BIM are subjected to chemical screenings to identify chemicals that induce more apoptosis in MEFs expressing MCL-IRES-BIM than wild-type MEFs. The identified chemicals include MCL-1 inhibitors that disrupt the interaction between MCL-1 and BIM and regulators of MCL-1 expression or protein stability. The same screening strategy can be performed in the isogenic H23 cancer cell lines with selective addiction to MCL-1, BCL-2, or BCL-X_{L} as shown in FIG. 3B.

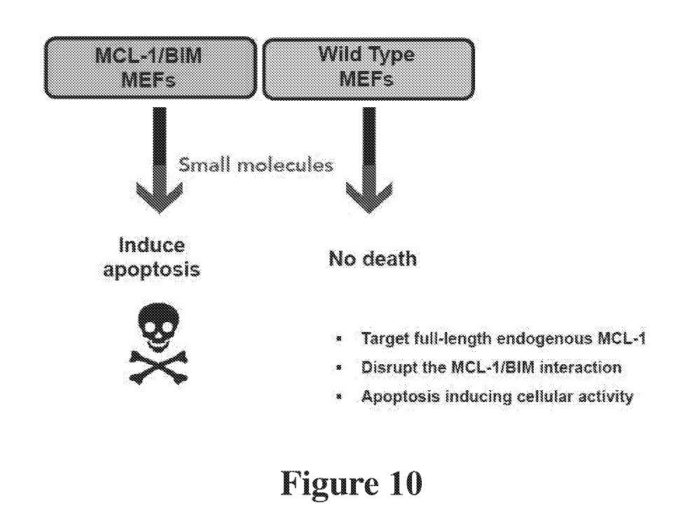

FIG. 11 shows three cell lines (DMS53, SW1417, H82) that were identified as having differential addiction to BCL-2, BCL-XL, and MCL-1. The structure of inhibitor F9 is shown in FIG. 3B.

### DETAILED DESCRIPTION OF CERTAIN EMBODIMENTS

New therapies based on inhibition of anti-apoptotic proteins are needed, and therefore new methods of screening potential inhibitors are crucially important. In one aspect, the present invention provides methods of engineering cells that mimic the “primed” cell death state of many cancers with a specific addiction to anti-apoptotic proteins for survival. The engineered cells can be used in methods for screening candidate inhibitors of the anti-apoptotic proteins, as described herein. In another aspect, the present invention provides cell lines and kits for practicing these methods. Once inhibitors of anti-apoptotic proteins are identified, these inhibitors may be used to treat diseases or disorders in a subject. Therefore, in yet another embodiment, the present invention provides methods for treating a disease in a subject using inhibitors of anti-apoptotic proteins. Certain embodiments of several aspects of the present invention follow.

### Methods for Screening Inhibitors

In one aspect, the present invention provides methods of engineering cells that mimic the “primed” cell death state of many cancers with a specific addiction to anti-apoptotic proteins for survival. In certain embodiments, the anti-apoptotic protein is a member of the BCL-2 family. In certain embodiments, the anti-apoptotic protein is selected from the group consisting of BCL-2, BCL-X_{L}, and MCL-1. In some embodiments, the method comprises expressing one or more anti-apoptotic BCL-2 member proteins in a cell. In certain embodiments, the BCL-2 member is selected from the group consisting of BCL-2, BCL-X_{L}, and MCL-1. In certain embodiments, the BCL-2 member is BCL-2. In certain embodiments, the BCL-2 member is BCL-X_{L}. In certain embodiments, the BCL-2 member is MCL-1. Any method known in the art can be used to express one or more anti-apoptotic BCL-2 member proteins in cells, including, but not limited to, transfection, plasmid-based expression, and viral vector expression. Expression of one or more proteins in the cells may be confirmed using any method known in the art, including, but not limited to, reporter gene assays, western blot, and ELISA (enzyme-linked immunosorbent assay).

In certain embodiments, the method comprises the steps of (a) expressing different combinations of anti-apoptotic BCL-2 members and activator BH3s in cells; and (b) converting the addiction of a cancer cell line to a specific anti-apoptotic BCL-2 member for survival to another anti-apoptotic BCL-2 member. In certain embodiments, the BCL-2 member expressed in the cell is selected from the group consisting of BCL-2, BCL-X_{L}, MCL-1, and combinations thereof. In certain embodiments, the cells are fibroblasts. In certain embodiments, the cells are mouse embryonic fibroblasts. In certain embodiments, the activator BH3 is BIM, PUMA or BID. In certain embodiments, the method comprises the steps of (a) expressing different combinations of anti-apoptotic BCL-2 members and activator BH3s such as BIM, PUMA and BID in mouse embryonic fibroblasts; and (b) converting the addiction of a cancer cell line to a specific anti-apoptotic BCL-2 member for survival to another anti-apoptotic BCL-2 member. In certain embodiments, the method comprises the steps of (a) expressing different combinations of BCL-2, BCL-X_{L}, and MCL-1, and activator BH3s such as BIM, PUMA and BID in mouse embryonic fibroblasts; and (b) converting the addiction of a cancer cell line to specific anti-apoptotic BCL-2 members (e.g., BCL-2 and/or BCL-X_{L}) for survival to an addiction to MCL-1.

As described herein, once one or more BCL-2 members are expressed in the cell, the addiction of the cancer cell for survival to another anti-apoptotic BCL-2 member can be effected. For example, in certain embodiments, the mouse embryonic fibroblasts expressing MCL-1-IRES-BIM or MCL-1-IRES-PUMA are addicted to MCL-1 for survival, the mouse embryonic fibroblasts expressing BCL-2-IRES-BIM or BCL-2-IRES-PUMA are addicted to BCL-2 for survival, and the mouse embryonic fibroblasts expressing BCL-X_{L}-IRES-BIM or BCL-X_{L}-IRES-PUMA are addicted to BCL-X_{L} for survival. In some instances, cells (e.g., H23, a K-RAS mutant lung cancer cell line) are dependent on MCL-1 for survival because knockdown of MCL-1 induces robust apoptosis, and its addiction to MCL-1 could be converted to BCL-2 or BCL-X_{L} addiction by overexpressing BCL-2 or BCL-X_{L} followed by knockdown of MCL-1. Likewise, in certain embodiments of the invention, addiction to BCL-2 and/or BCL-X_{L} can be converted to MCL-1 addiction by overexpression of MCL-1 followed by knockdown of BCL-2 and/or BCL-X_{L}.

In another aspect, the present invention provides methods of identifying inhibitors of anti-apoptotic survival pathways. In certain embodiments, the anti-apoptotic survival pathway involves overexpression of one or more member of the BCL-2 family (e.g., BCL-2, BCL-X_{L}, MCL-1). In certain embodiments, the anti-apoptotic survival pathway involves overexpression of MCL-1. In certain embodiments, the anti-apoptotic survival pathway involves overexpression of BCL-2. In certain embodiments, the anti-apoptotic survival pathway involves overexpression of BCL-X_{L}.

In certain embodiments, the method of identifying inhibitors comprises the steps of (a) exposing cultured wild-type cells to a candidate inhibitor at a predetermined concentration for a predetermined period of time and determining cell viability after the exposure to the candidate inhibitor; (b) exposing BCL-2 member protein addicted cells to the candidate inhibitor at the predetermined concentration for the predetermined period of time and determining cell viability after the exposure to the candidate inhibitor; and (c) identifying the candidate inhibitor as a BCL-2 family member inhibitor if the cell viability in step (a) is significantly higher than the cell viability in step (b).

In certain embodiments, determining cell viability involves measuring apoptosis. In certain embodiments, determining cell viability involves measuring caspase activity. In certain embodiments, determining cell viability involves measuring cytochrome c release. In certain embodiments, determining cell viability involves measuring cell membrane permeability.

In certain embodiments, the method of identifying inhibitors comprises the steps of (a) exposing cultured wild-type cells to a candidate inhibitor at a predetermined concentration for a predetermined period of time and determining cell viability after the exposure to the candidate inhibitor; (b) exposing MCL-1- or BCL-2-or BCL-X_{L}-addicted cells to the candidate inhibitor at the predetermined concentration for the predetermined period of time and determining cell viability after the exposure to the candidate inhibitor; and (c) identifying the candidate inhibitor as a MCL-1 or BCL-2 or BCL-X_{L} inhibitor if the cell viability in step (a) is significantly higher than the cell viability in step (b). In certain embodiments, the inhibitors identified by the inventive method are inhibitors of one or more members of the BCL-2 family (e.g., BCL-2, BCL-X_{L}, MCL-1).

The methods of identifying inhibitors described herein can be used to identify inhibitors that selectively inhibit a specific BCL-2 family member by employing two or more cell lines independently addicted to different members of the BCL-2 family. In certain embodiments, the method of identifying inhibitors comprises the steps of (a) exposing cultured wild-type cells to a candidate inhibitor at a predetermined concentration for a predetermined period of time and determining cell viability after the exposure to the candidate inhibitor; (b) exposing two or more cell lines independently addicted to MCL-1 or BCL-2 or BCL-X_{L} to the candidate inhibitor at the predetermined concentration for the predetermined period of time and determining cell viability after the exposure to the candidate inhibitor; and (c) identifying the candidate inhibitor as a MCL-1 or BCL-2 or BCL-X_{L} inhibitor if the cell viability in step (a) is significantly higher than the cell viability in step (b). In certain embodiments, the inhibitor is identified as a selective inhibitor of a specific BCL-2 family protein if the cell viability of one cell line of specifically addicted BCL-2 cells is significantly higher than the other cell lines of specifically addicted BCL-2 cells employed in step (b).

In certain embodiments, two cell lines independently and specifically addicted to two different BCL-2 family proteins are employed in step (b). In certain embodiments, two cell lines independently addicted to MCL-1 or BCL-2 or BCL-X_{L} are employed in step (b). In certain embodiments, one cell line in step (b) is specifically addicted to MCL-1, and the other cell line is specifically addicted to BCL-2. In certain embodiments, one cell line in step (b) is specifically addicted to MCL-1, and the other cell line is specifically addicted to BCL-X_{L}. In certain embodiments, one cell line in step (b) is specifically addicted to MCL-1, and the other cell line is addicted to BCL-2 and BCL-X_{L}. In certain embodiments, the candidate inhibitor is identified as a MCL-1 inhibitor if the BCL-2- and/or BCL-X_{L}-addicted cells show significantly higher cell viability than the MCL-1-addicted cells in step (b).

In certain embodiments, three cell lines independently and specifically addicted to different BCL-2 family proteins are employed in step (b). In certain embodiments, three cell lines independently addicted to MCL-1, BCL-2, and BCL-X_{L} are employed in step (b). In certain embodiments, the candidate inhibitor is identified as a MCL-1 inhibitor if the BCL-2- and or BCL-X_{L}-addicted cells show significantly higher cell viability than the MCL-1-addicted cells in step (b). In certain embodiments, more than three cell lines independently and specifically addicted to different BCL-2 family proteins are employed in step (b). The BCL-2 family proteins may be selected from the group consisting of BAK (BAK1), BAX, parent BCL-2, A1 (BCL2A1), BCL-XL (BCL2L1), BCL-W (BCL2L2), BCL-B (BCL2L10), BCL-RAMBO (BCL2L13), BCL-G (BCL2L14), BOK, and MCL-1.

As described herein, a candidate inhibitor is identified as a BCL-2 member inhibitor if the cell viability in step (a) of the method is significantly higher than the cell viability in step (b) of the method. In certain embodiments, the cells in step (a) are wild-type cells, the cells in step (b) are MCL-1 addicted cells, and the candidate inhibitor is identified as a MCL-1 inhibitor if the cell viability in step (a) is significantly higher than the cell viability in step (b). In certain embodiments, the cells in step (a) are wild-type MEFs, the cells in step (b) are MCL-1 addicted MEFs and the candidate inhibitor is identified as a MCL-1 inhibitor if the cell viability in step (a) is significantly higher than the cell viability in step (b).

In certain embodiments, the cells in step (a) are wild-type cells, the cells in step (b) are BCL-2 addicted cells, and the candidate inhibitor is identified as a BCL-2 inhibitor if the cell viability in step (a) is significantly higher than the cell viability in step (b). In some embodiments, the cells in step (a) are wild-type MEFs, the cells in step (b) are BCL-2 addicted MEFs and the candidate inhibitor is identified as a BCL-2 inhibitor if the cell viability in step (a) is significantly higher than the cell viability in step (b).

In certain embodiments, the cells in step (a) are wild-type cells, the cells in step (b) are BCL-X_{L}-addicted cells, and the candidate inhibitor is identified as a BCL-X_{L}inhibitor if the cell viability in step (a) is significantly higher than the cell viability in step (b). In certain embodiments, the cells in step (a) are wild-type MEFs, the cells in step (b) are BCL-X_{L}-addicted MEFs and the candidate inhibitor is identified as a BCL-X_{L} inhibitor if the cell viability in step (a) is significantly higher than the cell viability in step (b).

In certain embodiments, the method of screening inhibitors comprises the steps of (a) exposing cultured wild-type cells to a candidate inhibitor at a predetermined concentration for a predetermined period of time and determining cell viability after the exposure to the candidate inhibitor; (b) exposing MCL-1 addicted cells and BCL-2 or BCL-X_{L} addicted cells to the candidate inhibitor at the predetermined concentration for the predetermined period of time and determining cell viability after the exposure to the candidate inhibitor; and (c) identifying the candidate inhibitor as a MCL-1 inhibitor if the cell viability in step (a) is significantly higher than the cell viability in step (b). In certain embodiments, the method further comprises the step of (d) identifying the candidate inhibitor as a selective inhibitor of MCL-1 if the cell viability of the BCL-2- or BCL-X_{L}-addicted cells is significantly higher than the cell viability of the cell viability of the MCL-1-addicted cells in step (b).

In certain embodiments, the method of screening inhibitors comprises the steps of (a) exposing cultured wild-type cells to a candidate inhibitor at a predetermined concentration for a predetermined period of time and determining cell viability after the exposure to the candidate inhibitor; (b) exposing MCL-1 addicted cells and BCL-2 addicted cells to the candidate inhibitor at the predetermined concentration for the predetermined period of time and determining cell viability after the exposure to the candidate inhibitor; and (c) identifying the candidate inhibitor as a BCL-2 family inhibitor if the cell viability in step (a) is significantly higher than the cell viability in step (b). In certain embodiments, the method further comprises the step of (d) identifying the candidate inhibitor as a selective inhibitor of MCL-1 if the cell viability of the BCL-2 addicted cells is significantly higher than the cell viability of the cell viability of the MCL-1 addicted cells in step (b).

In certain embodiments, the method of screening inhibitors comprises the steps of (a) exposing cultured wild-type cells to a candidate inhibitor at a predetermined concentration for a predetermined period of time and determining cell viability after the exposure to the candidate inhibitor; (b) exposing MCL-1-addicted cells and BCL-X_{L}-addicted cells to the candidate inhibitor at the predetermined concentration for the predetermined period of time and determining cell viability after the exposure to the candidate inhibitor; and (c) identifying the candidate inhibitor as a BCL-2 family inhibitor if the cell viability in step (a) is significantly higher than the cell viability in step (b). In certain embodiments, the method further comprises the step of (d) identifying the candidate inhibitor as a selective inhibitor of MCL-1 if the cell viability of the BCL-X_{L}-addicted cells is significantly higher than the cell viability of the cell viability of the MCL-1-addicted cells in step (b).

In certain embodiments, the method of screening inhibitors comprises the steps of (a) exposing cultured wild-type cells to a candidate inhibitor at a predetermined concentration for a predetermined period of time and determining cell viability after the exposure to the candidate inhibitor; (b) exposing MCL-1-, BCL-X_{L}-, and BCL-2-addicted cells to the candidate inhibitor at the predetermined concentration for the predetermined period of time and determining cell viability after the exposure to the candidate inhibitor; and (c) identifying the candidate inhibitor as a BCL-2 family inhibitor if the cell viability in step (a) is significantly higher than the cell viability in step (b). In certain embodiments, the method further comprises the step (d) identifying the candidate inhibitor as a selective inhibitor of MCL-1 if the cell viability of the BCL-X_{L} addicted cells and BCL-2 addicted cells is significantly higher than the cell viability the MCL-1 addicted cells in step (b).

Cells used in the inventive methods (i.e., both wild-type cells and BCL-2 family member addicted cells) can be any type of cell. In certain embodiments, the wild-type cells are cancer cells. In certain embodiments, the wild-type cells are human cells. In certain embodiments, the addicted cells are cancer cells. In certain embodiments, the addicted cells are human cells. In certain embodiments, the wild-type cells are non-human animal cells. In certain embodiments, the addicted cells are non-human animal cells. In certain embodiments of the method, the wild-type cells are human cancer cells. In certain embodiments, the MCL-1- or BCL-2- or BCL-X_{L}-addicted cells are human cancer cells. In certain embodiments, the wild-type cells and the MCL-1- or BCL-2- or BCL-X_{L}-addicted cells are human cancer cells. In certain embodiments, the wild-type cells and the MCL-1-addicted cells are human cancer cells. In certain embodiments, the wild-type cells and the BCL-2-addicted cells are human cancer cells. In certain embodiments, the wild-type cells and the BCL-X_{L}-addicted cells are human cancer cells. In certain embodiments, the wild-type cells are isogenic human cancer cells. In certain embodiments, the MCL-1- or BCL-2- or BCL-X_{L}-addicted cells are isogenic human cancer cells. In certain embodiments, the wild-type cells and the MCL-1 addicted cells are isogenic human cancer cells. In certain embodiments, the wild-type cells and the BCL-2 addicted cells are isogenic human cancer cells. In certain embodiments, the wild-type cells and the BCL-X_{L} addicted cells are isogenic human cancer cells. In certain embodiments of the invention, the wild-type cells are embryonic fibroblasts. In certain embodiments, the MCL-1 or BCL-2 or BCL-X_{L} addicted cells are embryonic fibroblasts. In certain embodiments, the wild-type cells and MCL-1 or BCL-2 or BCL-X_{L} addicted cells are embryonic fibroblasts. In certain embodiments, the wild-type cells and MCL-1 addicted cells are embryonic fibroblasts. In certain embodiments, the wild-type cells and BCL-2 addicted cells are embryonic fibroblasts. In certain embodiments, the wild-type cells and BCL-X_{L} addicted cells are embryonic fibroblasts. In certain embodiments, the embryonic fibroblasts are mouse embryonic fibroblasts (MEFs). In certain embodiments of the invention, the wild-type cells are mouse embryonic fibroblasts. In certain embodiments, the MCL-1- or BCL-2- or BCL-X_{L}-addicted cells are mouse embryonic fibroblasts. In certain embodiments, the wild-type cells and MCL-1- or BCL-2- or BCL-X_{L}-addicted cells are mouse embryonic fibroblasts. In certain embodiments, the wild-type cells and MCL-1-addicted cells are mouse embryonic fibroblasts. In certain embodiments, the wild-type cells and BCL-2-addicted cells are mouse embryonic fibroblasts. In certain embodiments, the wild-type cells and BCL-X_{L}-addicted cells are mouse embryonic fibroblasts. In certain embodiments, the wild-type and/or MCL-1- or BCL-2- or BCL-X_{L}-addicted cells are K-RAS mutant cells. In certain embodiments, the wild-type cells are H23 parental cells. In certain embodiments, the MCL-1- or BCL-2- or BCL-X_{L}-addicted cells are H23 parental cells. In certain embodiments, the wild-type and the MCL-1- or BCL-2- or BCL-X_{L}-addicted cells are H23 parental cells. In certain embodiments, BCL-X_{L}-addicted cells are engineered H23 parental cells. Further examples of cell lines that can be used in the inventive method include, but are not limited to, A427 non-small cell lung cancer, H82 small cell lung cancer (SCLC), and DMS114 SCLC that are addicted to MCL-1, SK-LU-1 lung adenocarcinoma and SW1417 colorectal cancer cell lines that are addicted to BCL-X_{L}, and DMS53 SCLC that is addicted to BCL-2.

The candidate inhibitor screened in the inventive method can be any molecular agent. In certain embodiments, the candidate inhibitor is selected from the group consisting of small molecules, proteins, peptides, polymers, and nucleic acids. In certain embodiments, the candidate inhibitor is a protein. In certain embodiments, the candidate inhibitor is a peptide. In certain embodiments, the candidate inhibitor is a polymer. In certain embodiments, the candidate inhibitor is a small molecule. In certain embodiments, the candidate inhibitor is a therapeutic small molecule. In certain embodiments, the candidate inhibitor is a small molecule drug. In certain embodiments, the candidate inhibitor is an organic small molecule. In certain embodiments, the candidate inhibitor is an inorganic molecule. In certain embodiments, the candidate inhibitor is an organometallic molecule. In certain embodiments, the candidate inhibitor is a NOXA mimetic. In some embodiments, the candidate inhibitor down-regulates MCL-1 mRNA or protein. In some embodiments, the candidate inhibitor is a BAD mimetic. In some embodiments, the candidate inhibitor down-regulates BCL-2 mRNA or protein. In some embodiments, the candidate inhibitor is a BAD mimetic. In some embodiments, the candidate inhibitor down-regulates BCL-X_{L} mRNA or protein.

The candidate inhibitors may be screened via low-throughput screening (LTS) or high-throughput screening (HTS). In certain embodiments, the inventive method involves LTS of candidate inhibitors. In other embodiments, the method involves HTS of candidate inhibitors. In certain embodiments of the inventive method, cells expressing MCL-1-IRES-BIM or MCL-1-IRES-PUMA are addicted to MCL-1 for survival and can be utilized for HTS for MCL-1 inhibitors. In certain embodiments, the cells expressing BCL-2-IRES-BIM or BCL-2-IRES-PUMA are addicted to BCL-2 for survival and could be utilized for HTS for BCL-2 inhibitors. In certain embodiments, the cells expressing BCL-X_{L}-IRES-BIM or BCL-X_{L}-IRES-PUMA are addicted to BCL-X_{L} for survival and could be utilized for HTS for BCL-X_{L} inhibitors.

In certain embodiments, the MCL-1-addicted cells express higher levels of MCL-1 and BIM from a MCL-1-IRES-BIM construct. In certain embodiments, the MCL-1-addicted cells express higher levels of MCL-1 and PUMA from a MCL-1-IRES-PUMA construct. In some embodiments, the BCL-2-addicted cells express higher levels of BCL-2 and BIM from a BCL-2-IRES-BIM construct. In some embodiments, the BCL-2-addicted cells express higher levels of BCL-2 and PUMA from a BCL-2-IRES-PUMA construct. In some embodiments, the BCL-X_{L}-addicted cells express higher levels of BCL-X_{L} and BIM from a BCL-X_{L}-IRES-BIM construct. In some embodiments, the BCL-X_{L}-addicted cells express higher levels of BCL-X_{L} and PUMA from a BCL-X_{L}-IRES-PUMA construct.

As described herein, cell viability and/or extend of apoptosis can be measured by any method known in the art. Examples of methods for measuring cell viability and/or apoptosis include, but are not limited to, caspase activity assays, cytochrome c release assays, cell membrane permeability assays, fluorescent detection methods (e.g., live/dead cell viability assays), trypan blue assays, ATP tests, calcein AM assays, clonogenic assays, Evans blue assays, fluorescein diacetate hydrolysis/Propidium iodide staining, flow cytometry, formazan-based assays, green fluorescent protein assays, lactate dehydrogenase assays, methyl violet assays, Propidium iodide stain, Resazurin assays, and TUNEL assays.

In certain embodiments, the cell viability of wild-type cells is considered “significantly higher” than the cell viability of MCL-1- or BCL-2- or BCL-X_{L}-addicted cells if the cell viability of the wild-type cells is at least 1%, 5%, 10%, 20%, 40%, 50%, 60%, 70%, 80%, 90%, or 100% greater than the cell viability of the addicted cells. In certain embodiments, the cell viability of cells addicted to one or more specific BCL-2 members is “significantly higher” than the cell viability of cells addicted to other BCL-2 members if the cell viability of the first BCL-2 member-addited cells is at least 1%, 5%, 10%, 20%, 40%, 50%, 60%, 70%, 80%, 90%, or 100% greater than the cell viability of the second BCL-2 member-addicted cells. For example, the cell viability of BCL-2 and/or BCL-X_{L} addicted cells is “significantly higher” than the cell viability of MCL-1 addicted cells if the cell viability of the BCL-2 and/or BCL-X_{L} addicted cells is at least 1%, 5%, 10%, 20%, 40%, 50%, 60%, 70%, 80%, 90%, or 100% greater than the cell viability of the MCL-1 addicted cells. In certain embodiments, cell viability of one group of cells is “significantly higher” than cell viability of another group of cells if the cell viability of the first group of cells is more than 1-fold, not less than 2-fold, not less than 5-fold, not less than 10-fold, not less than 30-fold, not less than 100-fold, not less than 1,000-fold, or not less than 10,000-fold greater than the cell viability of the second group of cells.

In certain embodiments, the extent of apoptosis of MCL-1- or BCL-2- or BCL-X_{L}-addicted cells is considered “significantly higher” than the extent of apoptosis of wild-type cells if the extent of apoptosis of the MCL-1- or BCL-2- or BCL-X_{L}-addicted cells is at least 1%, 5%, 10%, 20%, 40%, 50%, 60%, 70%, 80%, 90%, or 100% greater than extent of apoptosis of the wild-type cells. In certain embodiments, the extent of apoptosis of cells addicted to one or more specific BCL-2 members is “significantly higher” than the cell viability of cells addicted to other BCL-2 members if extent of apoptosis of the second BCL-2 member-addited cells is at least 1%, 5%, 10%, 20%, 40%, 50%, 60%, 70%, 80%, 90%, or 100% greater than the extent of apoptosis of the second BCL-2 member-addicted cells. For example, the extent of apoptosis of MCL-1 addicted cells is “significantly higher” than the extent of apoptosis of the BCL-2 and/or BCL-X_{L} addicted cells if the extent of apoptosis the MCL-1 addicted cells is at least 1%, 5%, 10%, 20%, 40%, 50%, 60%, 70%, 80%, 90%, or 100% greater than the extent of apoptosis the BCL-2 and/or BCL-X_{L} addicted cells. In certain embodiments, extent of apoptosis of one group of cells is “significantly higher” than extent of apoptosis of another group of cells if the cell viability of the second group of cells is more than 1-fold, not less than 2-fold, not less than 5-fold, not less than 10-fold, not less than 30-fold, not less than 100-fold, not less than 1,000-fold, or not less than 10,000-fold greater than the extent of apoptosis of the first group of cells.

In certain embodiments, the cell viability of wild-type cells is considered “significantly higher” than the cell viability of MCL-1- or BCL-2- or BCL-X_{L}-addicted cells if the normalized caspase activity of the addicted cells is greater than the normalized caspase activity of the wild-type cells. In certain embodiments, the cell viability of wild-type cells is considered “significantly higher” than the cell viability of MCL-1 or BCL-2 or BCL-X_{L} addicted cells if the normalized caspase activity of the addicted cells is at least 1%, 5%, 10%, 20%, 40%, 50%, 60%, 70%, 80%, 90%, or 100% greater than the normalized caspase activity of the wild-type cells. In certain embodiments, the normalized caspase activity of the addicted cells is between 1% and 40% greater than the normalized caspase activity of the wild-type cells. In certain embodiments, the normalized caspase activity of the addicted cells is at least 40% greater than the normalized caspase activity of the wild-type cells Likewise, in certain embodiments, the cell viability of a specific BCL-2 member addicted cell is considered “significantly higher” than the cell viability of another BCL-2 member addicted cell if the normalized caspase activity of one cell line is at least 1%, 5%, 10%, 20%, 40%, 50%, 60%, 70%, 80%, 90%, or 100% greater than the other cell line. In certain embodiments, the normalized caspase activity is between 1% and 40% greater than the normalized caspase activity of the wild-type cells. In certain embodiments, the normalized caspase activity is at least 40% greater.

### Cell Lines and Kits

The present invention provides cell lines for carrying out the methods described herein. The cell lines may comprise any of the cells described herein, including wild-type and BCL-2 family protein addicted cells. In certain embodiments, the cell lines comprise any of the wild-type cells described herein. In certain embodiments, the cell lines comprise any of the BCL-2 family addicted cells described herein. In certain embodiments, the cell lines comprise BCL-2, MCL-1 or BCL-X_{L} addicted cells, or any combination thereof. In certain embodiments, the cell lines comprise MCL-1 addicted cells.

As described herein, cells used in the inventive methods (i.e., both wild-type cells and BCL-2 family member addicted cells) can be any type of cell. Examples of wild-type cells and BCL-2 member addicted cells include, but are not limited to, human cells (e.g., human cancer cells, isogenic human cancer cells) and non-human animal cells (e.g., embryonic fibroblasts, including, but not limited to, mouse embryonic fibroblasts). In certain embodiments, the cells include H23 parental cells. In certain embodiments, the cells include H23 parental cells engineered to express one or more members of the BCL-2 family (e.g., BCL-2, BCL-X_{L}, MCL-1)

Also provided herein are kits comprising cell lines for carrying out the inventive methods. The cell lines can be any of the cell lines described herein, which can comprise any of the cells described as being useful in the inventive methods. In certain embodiments, the kit comprises cell lines comprising wild-type cells and cell lines comprising BCL-2 member protein (e.g., BCL-2, MCL-1, BCL-X_{L}) addicted cells. In certain embodiments, the kit comprises cell lines comprising wild type cells and cell lines comprising MCL-1 addicted cells. Any of the kits described herein may further comprise instructions for performing or executing the methods described herein. Any of the kits may further comprise one or more candidate inhibitors for screening using the inventive method. In certain embodiments, the kit further comprises control compounds. In certain embodiments, the kit further comprises buffers useful for practicing the inventive method described herein. In certain embodiments, the kit comprises instructions for practicing the inventive method described herein.

### Methods for the Treatment of Disorders

In another aspect, the present invention provides methods for treating diseases or disorders with a BCL-2 family inhibitor. In certain embodiments, the present invention provides methods for treating diseases or disorders with an agent identified by the inventive screening method. In certain embodiments, the disease or disorder is associated with defective apoptosis (e.g., cancer, arthritis, inflammation, lymphoproliferative conditions, inflammatory diseases, and autoimmune diseases). In certain embodiments, the disease is an inflammatory disease, an autoimmune disease, a proliferative disease. In certain embodiments, the disease is a neoplasm or tumor. In certain embodiments, the disease is associated with angiogenesis. In certain embodiments, the disease is cancer In certain embodiments, the method comprises the step of administering to a subject in need thereof an effective amount of an MCL-1 or BCL-2 or BCL-X_{L} inhibitor, or a pharmaceutical composition thereof. In certain embodiments, the method for treating cancer in a subject comprises administering to a subject in need thereof an effective amount of a MCL-1 inhibitor, or a salt thereof, or a pharmaceutical composition thereof. In certain embodiments, the method for treating cancer in a subject comprises administering to a subject in need thereof an effective amount of a selective MCL-1 inhibitor, or a salt thereof, or a pharmaceutical composition thereof, wherein a “selective MCL-1 inhibitor” is an inhibitor that targets MCL-1 and not other members of the BCL-2 family (e.g., BCL-2, BCL-X_{L}).

In other embodiments, the MCL-1 inhibitor is administered in combination one or more additional agents. In certain embodiments, the additional agent is a therapeutic agent. In certain embodiments, the additional agent is an anti-cancer agent, wherein “anti-cancer agent”is as defined herein. In some embodiments, the second agent is a BCL-2 or BCL-X_{L} inhibitor, or a pharmaceutical composition thereof. In some embodiments, the second agent is ABT-737 or ABT-263, or a pharmaceutical composition thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Pharmaceutical compositions described herein may comprise one or more MCL-1, BCL-2, or BCL-X_{L} inhibitors, and optionally a pharmaceutically acceptable excipient. Pharmaceutical compositions described herein may further comprise one or more additional therapeutic agents. Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Certain modes for carrying out the invention are presented in terms of exemplary embodiments, discussed herein. However, the application is not limited to the described embodiments and a person skilled in the art will appreciate that many other embodiments of the application are possible without deviating from the basic concept of the application, and that any such work around will also fall under scope of this application. It is envisioned that other styles and configurations of the present application can be easily incorporated into the teachings of the present application, and only one particular configuration shall be shown and described for purposes of clarity and disclosure and not by way of limitation of scope.

### EXAMPLES

In order that the invention described herein may be more fully understood, the following examples are set forth. The examples provided in this application are offered to illustrate the methods, cell lines, and kits provided herein and are not to be construed in any way as limiting their scope.

### Development and Empolyment of a Cell-Based Screening Strategy for MCL-1 Inhibitors

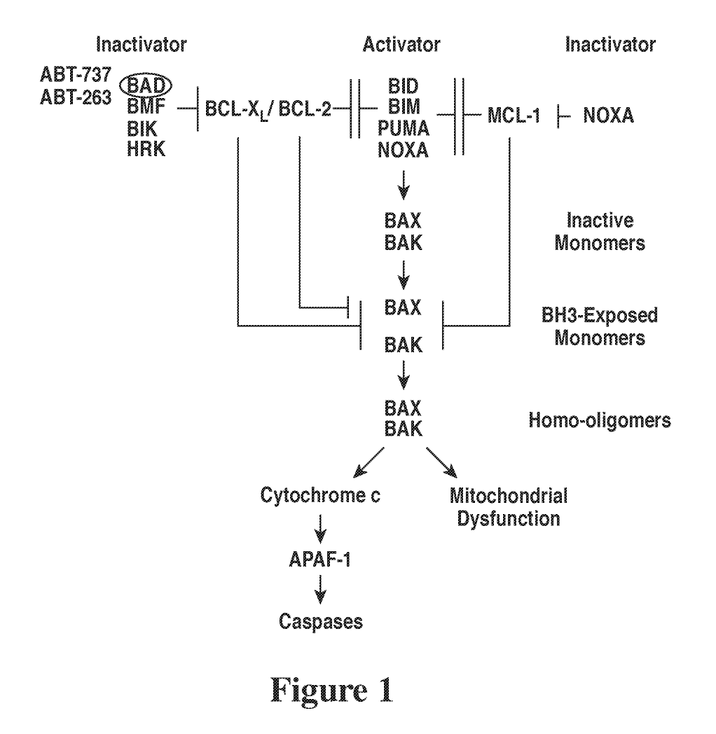

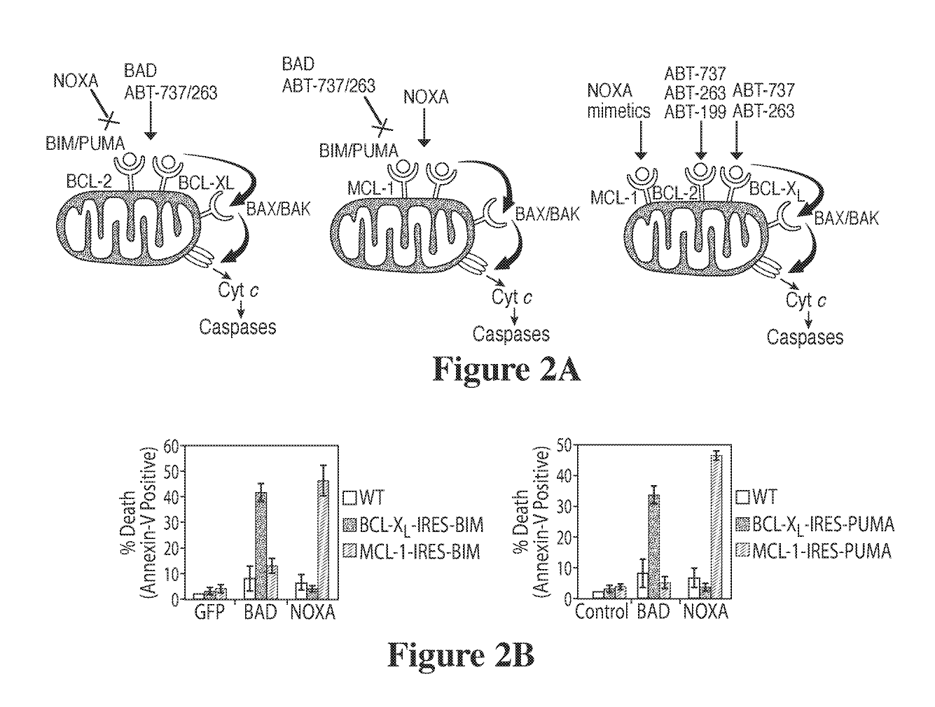

Cellular dependency on BCL-2, BCL-X_{L} or MCL-1 for survival is governed by the relative abundance among these proteins (FIG. 1). Intricate interplays among the BCL-2 subfamilies govern cellular survival/death, and also provide a molecular blueprint concerning the clinical application of BH3-mimetics in killing cancer cells. Using MEFs that express different combinations of anti-apoptotic BCL-2 members and activator BH3s, a system has been built that can distinguish between the inhibition of BCL-2/BCL-X_{L} and MCL-1 by BAD/BAD mimetics and MOXA/NOXA mimetics, respectively (FIGS. 2A and 2B). Because NOXA can only displace BIM or PUMA from MCL-1, but not from BCL-2/BCL-X_{L} to activate BAX/BAK, NOXA selectively induces apoptosis in MCL-1-IRES-BIM or MCL-1-IRES-PUMA but not BCL-X_{L}-IRES-BIM, BCL-X_{L}-IRES-PUMA, BCL-2-IRES-BIM, or BCL-2-IRES-PUMA MEFs (FIGS. 2A and 2B).

In contrast, BAD induces apoptosis in BCL-X_{L}-IRES-BIM, BCL-X_{L}-IRES-PUMA, BCL-2-IRES-BIM or BCL-2-IRES-PUMA cells but not MCL-1-IRES-BIM or MCL-1-IRES-PUMA cells because BAD binds to BCL-X_{L} and BCL-2 but not MCL-1 (FIGS. 2A and 2B). Importantly, this system mimics the “primed” cell death state of many cancers with abundant pre-assembled complexes of BCL-X_{L}/BIM, BCL-X_{L}/PUMA, BCL-2/BIM, BCL-2/PUMA, MCL-1/BIM or MCL-1/PUMA. Conversely, wild-type cells are less sensitive to BAD or NOXA due to the lack of pre-assembled cell death priming complexes (FIG. 2B). Based on these data, NOXA-mimetics will trigger more apoptosis in MCL-1-IRES-BIM than wild-type cells. In addition, compounds that downregulate MCL-1 mRNA and/or protein or induce endogenous NOXA, BIM, or PUMA will trigger similar death patterns.

Low-throughput screens using the NCI DTP (Developmental Therapeutics Program) Diversity Set 1,900 compounds and the ChemBridge DiverSet A (10,000 compounds) are used to identify compounds that display more than 20% growth-inhibitory effect in MCL-1-IRES-BIM than wild-type MEFs. Compounds are identified that trigger more apoptosis in MCL-1-IRES-BIM or MCL-1-IRES-PUMA than BCL-X_{L}-IRES-BIM, BCL-X_{L}-IRES-PUMA, BCL-2-IRES-BIM, BCL-2-IRES-PUMA or wild-type cells.

### Identification of MCL-1-Addicted Cancer Cell Lines and Assessment of Small Molecule Inhibitors of MCL-1 Discovered in Pilot Screens

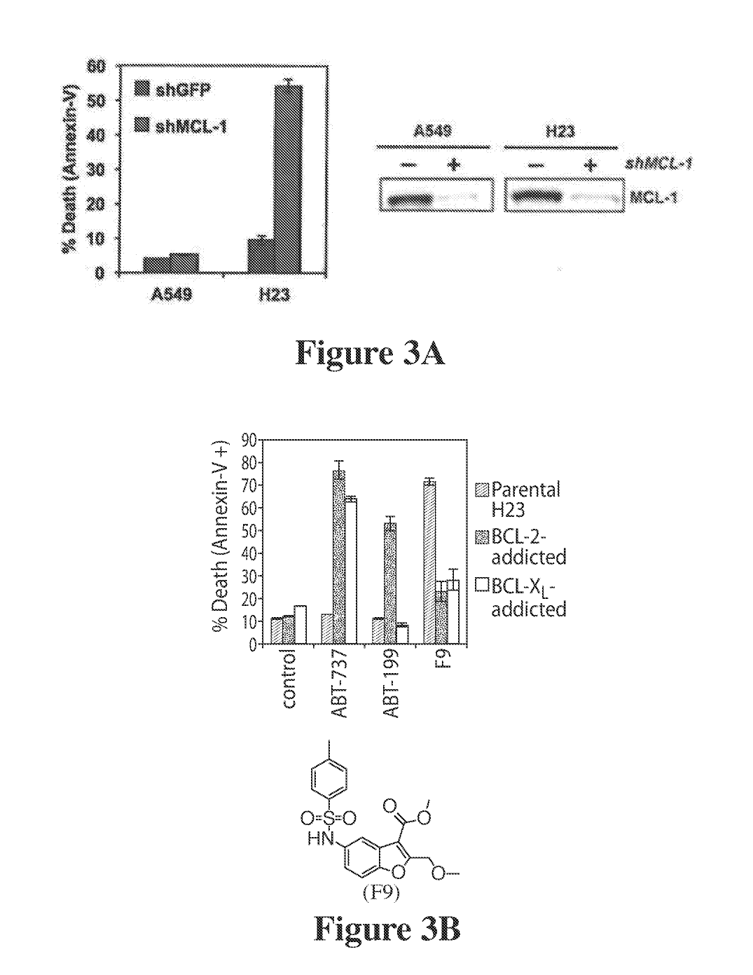

H23, a K-RAS mutant lung cancer cell line, requires MCL-1 for survival due to its high MCL-1 and low BCL-2/BCL-X_{L} expression (FIG. 3A). In contrast, knockdown of MCL-1 in A549, another K-RAS mutant lung cancer cell line, induces minimal apoptosis. Nevertheless, knockdown of MCL-1 renders A549 cells susceptible to ABT-737-induced apoptosis because concurrent inhibition of BCL-2, BCL-X_{L} and MCL-1 is required to activate BAX/BAK in A549 cells.

Candidate compounds that specifically antagonize MCL-1 will trigger apoptosis in H23 cells as a single agent and synergize with ABT-737 to trigger apoptosis in A549 cells. Of note, the synergistic effect is absent in Bax^{-/-}Bak^{-/-} cells, confirming the activation of BAX/BAK. Since both H23 and A549 cell lines display similar EC50 for paclitaxel, the selective sensitivity of H23 to these compounds is not simply due to a death-prone phenotype of H23 cells.

### Characterization of Small Molecule Inhibitors of MCL-1 Identified in Pilot Screens

There are three potential mechanisms by which compounds can inhibit the pro-survival function of MCL-1. They can (1) directly bind and inhibit the hydrophobic binding groove of MCL-1 as NOXA mimetics; (2) downregulate MCL-1 through transcriptional/translational/post-translational mechanisms; or (3) induce NOXA, BIM or PUMA. In some embodiments, compounds reduce MCL-1 protein by effectively inducing apoptosis in MCL-1-addicted H23 cancer cells. In particular embodiments, a compound will bind to the hydrophobic dimerization pocket of MCL-1. In other embodiments, a compound does not bind to MCL-1 and may reduce MCL-1 protein stability by either inhibiting the deubiquitinases of MCL-1 or activating the E3 ligases of MCL-1.

### Establishment of and Performance of Cell-Based High-Throughput Screening to Identify Inhibitors of the MCL-1-Dependent Survival Pathway for Cancer Therapy

A. Establishment of Isogenic Cancer Cell Lines that are Selectively Cancer Cell Lines that are Selectively Addicted to BCL-2, BCL-X_{L} or MCL-1 for Mechanistic Studies and High-Throughput Screening for MCL-1 Inhibitors

Pilot screens using engineered MEFs mimicking “primed” cell death state of cancers have led to the identification of mechanism-specific compounds. Hence, BCL-2, BCL-X_{L} or MCL-1 singularly addicted isogenic cancer cell lines are generated to further validate the specificity of hit compounds against MCL-1 and for additional high-throughput screening (HTS).

K-RAS mutant H23 cell lines have been converted from MCL-1 addiction to BCL-2 or BCL-X_{L} addiction by overexpressing BCL-2 or BCL-X_{L} followed by knockdown of MCL-1. The selective addiction of engineered H23 cells was confirmed by treating these cell lines with inhibitors of BCL-2 (ABT-737 and ABT-199), BCL-X_{L} (ABT-737), and MCL-1 (F9). As expected, the BCL-2-addicted H23 cells are sensitive to ABT-737 and ABT-199 but not inhibitors of MCL-1, the BCL-X_{L}-addicted H23 cells are sensitive to ABT-737 but not ABT-199 or inhibitors of MCL-1, and parental H23 cells are only sensitive to inhibitors of MCL-1 (FIG. 3B).

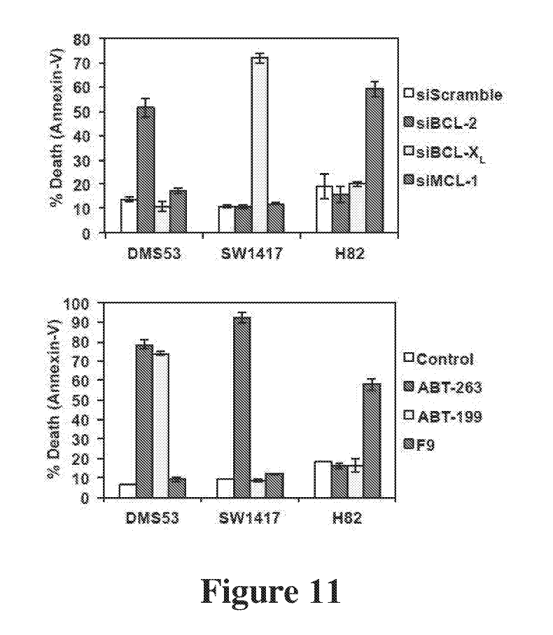

To extend the study to different cancer types harboring distinct driver mutations, the Broad Novartis Cancer Cell Line Encyclopedia was mined to identify cancer cell lines that highly express one anti-apoptotic BCL-2 member and confirm their respective dependency using RNA interference technology. Thus far, A427 non-small cell lung cancer, H82 small cell lung cancer (SCLC), and DMS114 SCLC that are addicted to MCL-1, SK-LU-1 lung adenocarcinoma and SW1417 colorectal cancer cell lines that are addicted to BCL-X_{L}, and DMS53 SCLC that is addicted to BCL-2 have been identified. The dependency of SK-LU-1 and SW1417 cell lines to BCL-X_{L} is converted to MCL-1 addiction by overexpression MCL-1 followed by knockdown of BCL-X_{L}. FIG. 11 shows three cell lines (DMS53, SW1417, H82) that were identified as having differential addiction to BCL-2, BCL-XL, and MCL-1. These cell lines can be employed to determine the specificity of candidate compounds against MCL-1 versus BCL-2 /BCL-X_{L}. More importantly, the MCL-1-addicted, BCL-2 or BCL-X_{L}-addicted isogenic cancer cell lines will be utilized for HTS proposed below.

B. Establishment of a Cell-Based High-Throughput Screening Platform to Identify Inhibitors of the MCL-1-Dependent Survival Pathway with Defined Mechanisms of Action

Pilot screens demonstrated that the proposed assay platform is able to identify mechanism-specific compounds with cellular activity. Herein, low-throughput Alamar Blue assays are adapted to high-throughput Caspase-Glo assays for higher sensitivity and specificity. CellTiter-Glo assays can be alternatives to Caspace-Glo assays for determining cell viability. Parallel screening is performed on wild-type and MCL-1-IRES-BIM expressing MEFs to identify chemicals that selectively induce apoptosis in MCL-1-IRES-BIM but not wild-type cells, which is the same approach as for the low-throughput screens. Parallel HTS is performed using MCL-1- and BCL-X_{L}-addicted isogenic H23 cancer cells. The BCL-X_{L}-addicted are chosen over the BCL-2-addicted cell lines as a control based on the fact that both MCL-1 and BCL-X_{L} bind BAK with high affinity whereas BCL-2 preferentially interacts with BAX. Moreover, a promising selective BCL-2 inhibitor ABT-199 is currently in clinical trials. In contrast, no clinically applicable BCL-X_{L}-specific inhibitor is available. These screens may also identify BCL-X_{L} specific inhibitors.

A library of over 300,000 diverse compounds, and two sets of cell lines, were screened. The first set includes wild-type MEFs and MEFs stably expressing MCL-1-IRES-BIM, which has been used in the pilot screens for the discovery of promising leads. The second set includes H23 parental cell line and the engineered BCL-X_{L}-addicted H23 cell line. The HTS is performed in 384-well plates and the viability of cells is determined by caspase activity. Accordingly, Caspase-Glo® 3/7 assays (Promega) are used to quantify effector caspase activation, which is more specific for apoptosis and at the same time provides a wide dynamic range.

The HTS assays are optimized by determining the cell seeding density, pre-treatment seeding time, compound treatment time, and DMSO tolerance (vehicle). Compounds are screened at 10 μM concentration (0.2% DMSO). The relative caspase activity is expressed as the ratio of the luminescence signal of a compound-treated well minus the luminescence signal of a negative control well (0.2% DMSO) to the luminescence signal of a positive control well (staurosporine) minus the luminesce signal of a negative control. The hit criteria will be based on the relative activity of the sample compound versus intraplate positive (F9 at EC80 concentration) and negative (DMSO only) controls. If CellTiter-Glo assays are employed, the effect of compounds on viability will be expressed as percentage growth inhibition compared to positive (staurosporine) and negative controls (0.2% DMSO) using the following equation: % inhibition=((negative control average−read value of a compound-treated well)/(negative control average−positive control average))×100. A compound will be considered a “hit” if it induces≥2-fold growth inhibition in MCL1-IRES-BIM MEFs than WT MEFs. A statistically significant cutoff based on the z-score will be applied and the selected primary hits will be picked and tested in full 11-point concentration response experiments and in parallel secondary assays and analyzed in HPLC-MS for purity and structural integrity.

The hits identified in both MEFs and H23 cells represent the most specific inhibitors of the MCL-1-dependent survival pathway. Furthermore, BCL-X_{L}-specific inhibitors may be identified that induce more apoptosis in the BCL-X_{L}-addicted H23 cells than parental H23 cells.

C. Performance of Secondary Screening to Validate Apoptosis Induction through the Inhibition of MCL-1-Dependent Survival Pathway

The specificity of hit compounds in MCL-1 inhibition is confirmed by comparing their death-inducing effect in cell lines with selective addiction to MCL-1, BCL-X_{L} or BCL-2 using annexin-V assays. Cytochrome c translocation, a hallmark of mitochondrial outer membrane permeabilization, is also assessed. Lastly, it is confirmed that these compounds do not have any effect on Mcl-1 KO MEFs.

It has been confirmed that hit compounds induce apoptosis in MCL-1-IRES-BIM and MCL-1-IRES-PUMA MEFs but not in wild-type, BCL-X_{L}-IRES-BIM, BCL-X_{L}-IRES-PUMA, BCL-2-IRES-BIM or BCL-2-IRES-PUMA MEFs. Cell viability is quantified by FACS analysis following annexin-V staining. Hit compounds that induce more apoptosis in MCL-1-addicted MEFs are further assessed for their ability in triggering cytochrome c translocation by immunofluorescence. The same assays are employed to confirm that hit compounds induce apoptosis in MCL-1-addicted but not BCL-2- or BCL-X_{L}-addicted cancer cells. Along the same lines, it is determined whether the hit compounds synergize with ABT-737 or ABT-263 to induce apoptosis in A549 or wild-type MEFs that are not addicted to a single anti-apoptotic BCL-2 member for survival.

Finally, it is confirmed that the synergistic effect of hit compounds with ABT-737 or ABT-263 is dependent on MCL-1 using Mcl-1 KO MEFs and on BAX/BAK-dependent apoptosis using Bax^{-/-}Bak^{-/-} MEFs. The activity of hit compounds is further assessed by determining their EC50 in killing H23 cells.

Additional MCL-1 inhibitors with defined mechanisms of action are identified, which include chemicals that downregulate MCL-1 mRNA, target MCL-1 for degradation or induce endogenous BH3-only proteins. Mcl-1 KO MEFs are instrumental in differentiating NOXA mimetics from chemicals that target MCL-1 for degradation.

### D. Characterization of Identified Small Molecule Inhibitors of the MCL-1-Dependent Cancer Cell Survival Pathway

If hit compounds are NOXA mimetics, they will bind to the hydrophobic dimerization pocket of MCL-1 to displace BIM. First, hit compounds will disrupt the co-immunoprecipitation of MCL-1 and BIM in cells. Second, potential interactions between candidate compounds and MCL-1 are determined using surface plasmon resonance (SPR) assays. The equilibrium dissociation constant (K_{D}, binding constant) is calculated from the association (kₐ, on rate) and dissociation rates (k_{d}, off rate). An inhibitor of MCL-1 or BIM BH3 peptides will serve as positive controls. Recombinant MCL-1 proteins carrying mutations in the hydrophobic dimerization groove (W261A/G262A/R263A) that disrupt the heterodimerization between MCL-1 and BIM are also be included for comparison. A ProteOn™ XPR36 instrument (Bio-Rad) is used in these assays.

Third, the ability of compounds in disrupting the binding of FITC-labeled BIM BH3 peptides from recombinant MCL-1 protein is directly assessed using fluorescence polarization assays (FPA) to determine dissociation constants (K₁). Recombinant BCL-X_{L} and BCL-2 proteins are included for comparison in both SPR and FPA.

Bona fide NOXA mimetics are identified that display specific interaction with MCL-1 but not BCL-2 or BCL-X_{L}. Noteworthy, the binding affinity of NOXA mimetics to truncated MCL-1 protein in vitro may not reflect their interaction in cells.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of identifying MCL-1 or BCL-2 or BCL-X_{L} inhibitors, the method comprising:
(a) exposing cultured wild-type cells to a candidate inhibitor at a predetermined concentration for a predetermined period of time and determining cell viability after the exposure to the candidate inhibitor;
(b) exposing two or more cell lines independently addicted to MCL-1 or BCL-2 or BCL-X_{L} to the candidate inhibitor at the predetermined concentration for the predetermined period of time and determining cell viability after the exposure to the candidate inhibitor; and
(c) identifying the candidate inhibitor as a MCL-1 or BCL-2 or BCL-X_{L} inhibitor if the cell viability in step (a) is significantly higher than the cell viability in step (b).

2. The method of claim 1, further comprising the step of (d) identifying the candidate inhibitor as a selective inhibitor MCL-1 or BCL-2 or BCL-X_{L} if the cell viability of one of the cell lines in step (b) is significantly higher than the cell viability of the other cell lines in step (b).

3. The method of claim 1, wherein step (b) comprises exposing the candidate inhibitor to MCL-1 addicted cells and BCL-2 addicted cells.

4. The method of claim 1, wherein step (b) comprises exposing the candidate inhibitor to MCL-1 addicted cells and BCL-X_{L} addicted cells.

5. The method of claim 1, wherein step (b) comprises exposing the candidate inhibitor to MCL-1, BCL-2, and BCL-X_{L} addicted cells.

6. The method of claim 1, whrein the candidate inhibitor is identified as a MCL-1 inhibitor if the cell viability in step (a) is significantly higher than said cell viability of the MCL-1 addicted cells in step (b).

7. The method of claim 1, wherein the candidate inhibitor is identified as a selective MCL-1 inhibitor if the cell viability of BCL-2 and/or BCL-X_{L} addicted cells in step (b) is greater than the cell viability of MCL-1 addicted cells in step (b).

8. The method of claim 1, wherein one of the cell lines in step (b) comprise MCL-1 addicted cells, and wherein the MCL-1 addicted cells express higher levels of MCL-1 and BIM than the wild-type cells.

9. The method of claim 8, wherein the higher levels of MCL-1 and BIM are expressed from a MCL-1-IRES-BIM construct.

10. The method of claim 1, wherein the candidate inhibitor is a small molecule.

11. (canceled)

12. The method of claim 1, wherein the candidate inhibitor is a NOXA mimetic.

13. The method of claim 1, wherein the candidate inhibitor is a BAD mimetic.

14. The method of claim 1, wherein the wild-type cells and addicted cells are independently embryonic fibroblasts.

15. The method of claim 14, wherein the wild-type cells and addicted cells are independently mouse embryonic fibroblasts.

16. The method of claim 1, wherein the wild-type cells and addicted cells are independently human cells.

17. The method of claim 16, wherein the wild-type cells and the addicted cells are independently human cancer cells.

18. A kit comprising:
wild type cells;
two or more cell lines, wherein each cell line comprises MCL-1 or BCL-2 or BCL-X_{L} addicted cells; and
optionally instructions for performing the method of claim 1.

19. A method of treating a disease in a subject, the method comprising:
administering to the subject a therapeutically effective amount of selective MCL-1 inhibitor, or a pharmaceutically acceptable salt thereof, or a pharmaceutically acceptable composition thereof.

20. The method of claim 19, wherein the disease is cancer.

21. The method of claim 19, wherein the subject is a human.
